(19) United States
(12) Patent Application Publication
(10) Pub. No.: US 2016/0022751 A1
(43) Pub. Date: Jan. 28, 2016

(54) NOVEL COMPOSITION FOR TREATING ALZHEIMER'S DISEASE AND IMPROVING COGNITIVE FUNCTION OF ALZHEIMER'S PATIENTS

(71) Applicant: GANGNEUNG-WONJU NATIONAL UNIVERSITY INDUSTRY ACADEMY COOPERATION GROUP, Gangwon (KR)

(72) Inventor: Seong-Soo JOO, Gyeonggi-do (KR)

(73) Assignee: GANGNEUNG-WONJU NATIONAL UNIVERSITY INDUSTRY ACADEMY COOPERATION GROUP, Gangwon (KR)

(21) Appl. No.: 14/640,906

(22) Filed: Mar. 6, 2015

(30) Foreign Application Priority Data
Jul. 23, 2014 (KR) 10-2014-0093282
Dec. 31, 2014 (KR) 10-2014-0194881

(51) Int. Cl. A61K 36/258; A61K 31/704

(52) U.S. Cl. CPC A61K 36/258; A61K 31/704

(57) ABSTRACT

The present invention relates to a pharmaceutical composition for treating Alzheimer's disease and improving cognitive function of Alzheimer's patients, and more specifically for pharmaceutical composition for treating Alzheimer's disease and improving cognitive function of Alzheimer's patients comprising ginsenoside complex prepared by sequential fraction of steam-dried unripened ginseng berries using solvents and used thereof.

### CROSS REFERENCES

This application claims the benefit of Korean Patent Application Nos. 10-2014-0093282 and 10-2014-0194881, filed Jul. 23, 2014 and Dec. 31, 2014, respectively, the contents of each of which are incorporated herein by reference.

### TECHNICAL FIELD

The present invention relates to a novel pharmaceutical composition and more particularly to a composition for the treatment and improve cognitive function in Alzheimer's patients.

Neurodegenerative diseases, such as Alzheimer's disease (AD) or Parkinson's disease (PD), are incurable and debilitating conditions that result in progressive degeneration and/or death of nerve cells that primarily affect the neurons in the human brain (Esposito and Cuzzocrea, Curr. Med. Chem., 17: 2764-2774, 2010). Clinically, AD is characterized by low amounts of the enzyme choline acetyltransferase (ChAT) and enzyme abnormalities, which would produce reduced levels of the neurotransmitter acetylcholine (ACh) (Giacobini E. Int. J. Geriatr. Psychiatry, 18:S1-S5, 2003). ChAT activity and expression have been considered as markers for cholinergic neurons, which are involved in learning, memory, arousal, sleep, and movement (Oda Y., Pathol. Int., 49: 921-937, 1999). Vesicular acetylcholine transporter (VAChT), which is responsible for the translocation of cytoplasmic acetylcholine synthesized by the catalytic activity of ChAT and involved in memory loss, is also specifically required for cholinergic neurotransmission (Roghani and Carroll, Mol. Brain Res., 100: 21-30, 2002; Chen et al., J. Alzheimers Dis., 26: 755-766, 2011). Coexpression of these two proteins, which are colocalized within the genome and coregulated by various neurotrophic factors in neurons, is coordinately regulated and known to contribute significantly to the increase of intracellular Ach (Berse and Blusztajn, J. Biol. Chem., 270: 22101-22104, 1995). Thus, expression of ChAT and of VAChT is required for the acquisition and the maintenance of the cholinergic phenotype after the induction of differentiation.7 (Yamamuro and Aizawa, Life Sci., 86: 839-843, 2010).

Our previous study demonstrated that ChAT overexpressing human neural stem cells restored cognition in AF64A cholinotoxin rat model, resulting in an increasing ACh level in rat cerebrospinal fluid (CSF) (Park et al., Exp. Neurol., 234: 521-526, 2012). This implies that the increase of the ChAT level in cholinergic neurons plays a critical role in cognition enhancement and thus the cholinergic gene locus “VAChT/ChAT” can be a putative target for therapeutic approaches on AD (Nah et al., CNS Drug Rev., 13: 381-404, 2007; Chang et al., Eur. J. Pharmacol., 578: 28-36, 2008).

In addition, Alzheimer's dementia is disease that occurs most frequently in the aged population and about 10% of the population of 65-85 years of age and about 40% of the population over 85 years of age suffers this disease. Alzheimer's dementia was reported at first by the observation of Alois Alzheimer of Germany in 1907. He observed that nerve cells of hippocampus and neocortex of the brain of Alzheimer's patients were lost and there were abnormal structures such as neurofibrillary tangles (NTFs) which look like tangled bundles of fiber and senile plaques within the cell body of neurons. Senile plaques are extracellular deposits of amyloid peptide surrounded by neurites astrocytes, and microglia and are found mainly in the limbic structure or association neocortex. Nerve fiber clump can be said as an insoluble structure having a form of double paired twisted linear fibers formed by hyper-phosphorylated tau proteins.

Many studies have been conducted to investigate whether these abnormal tissue properties relate to the pathogenesis of Alzheimer's dementia and a hypothesis that the formation of amyloid beta (Aβ) plaque which is a main component of plaque would serve as the main reason for the onset of Alzheimer's dementia has been received the most attention. Aβ protein has been known to reduce activity of acetylcholine, a neurotransmitter by destructing neurons of the cerebral cortex synthesizing the acetylcholine and to result in neuronal death by generating reactive oxygen species (ROS) and causing oxidative damage thereby. Mechanisms involved in inflammation as well as the oxidative cell death are major causes leading to the damage of neurons.

U.S. Pat. No. 6,083,932A discloses pharmaceutical compositions comprising ginseng extract whose total saponin fraction is about 20-50% by weight of the ginseng extract and a method of improving learning ability or memory in a patient using the pharmaceutical composition.

### Technical Problems

However, it is not clearly identified whether what kind of components of ginseng are effective in the improvement of cognitive function of patients including Alzheimer's patients. The present invention has been made to solve a variety of problems, including the above-mentioned problems, to provide a composition effective for more improving cognitive function of Alzheimer's patients. However, the purpose is to be illustrative and the scope of the invention is not limited thereto.

### SUMMARY OF THE INVENTION

In an aspect of the present invention, a method for preparing ginsenoside complex effective for Alzheimer's disease is provided. The method comprises: i) preparing steam-dried unripened ginseng berries by steaming and drying unripened ginseng berries 3 to 10 times; ii) preparing extract of steam-dried ginseng berries by extracting the streamed-dried ginseng berries by extracting the steamed-dried ginseng berries with water, C1 to C4 alcohol or mixture thereof; iii) fractionating the extract of steam-dried ginseng berries by adding solvent comprising ethyl acetate and water to the extract of steam-dried ginseng berries and fractionating to ethyl acetate layer and water layer; and iv) preparing n-butanol fraction by further fractionating the water layer with n-butanol, and forming n-butanol layer thereby and recrystallizing the n-butanol layer with ether.

According to the method, the alcohol may be methanol, ethanol or propanol.

According to the method, the ginsenoside complex may comprise at least 4 wt % of ginsenoside Re, at least 8 wt % of ginsenoside Rd and at least 20 wt % of ginsenoside Rg3.

The method may further comprise isolating fractions corresponding Rf value of 0.4 to 0.7 on thin layer chromatography (TLC) using running solvent of ethyl acetate:methanol=3:1 from eluted fractions of the n-butanol fraction by a chromatography.

In another aspect of the present invention, a pharmaceutical composition for treating Alzheimer's disease and improving cognitive function of Alzheimer's patient comprising the ginsenoside complex prepared by the above-described method and at least a pharmaceutically acceptable carriers is provided.

In another aspect of the present invention, a nutraceutical composition for improving cognitive function of a subject comprising the ginsenoside complex prepared by the above-described method and at least a food additive is provided.

In still another aspect of the present invention, a method for treating and improving cognitive function of a subject suffering Alzheimer's disease comprising administrating a therapeutically effective amount of ginsenoside complex prepared by the above-described method to a subject suffering Alzheimer's disease is provided.

Ginsenoside complex containing ginsenoside Rd, Re and Rg3 according to one embodiment of the present invention prepared as described above may be used for treating Alzheimer's disease and for improving cognitive function of Alzheimer's patients since it can pass through the blood-brain barrier (BBB) as well as upregulate the expression of ChAT and AChE related to cognitive function and degrade amyloid beta, a causative substance for neuronal cell death of hippocampus and cerebral cortex.

### BRIEF DESCRIPTION OF DRAWINGS

FIG. 1 is a schematic diagram of the extraction process of ginsenoside-containing fraction or ginsenoside complex (hereinafter, referred to “RgC”) from steam-dried ginseng berries in accordance with an embodiment of the present invention.

FIG. 2 is a graph showing an HPLC chromatogram of RgC depending on the number of steam-drying according to an embodiment of the present invention.

FIG. 3 is a photographic image representing a result of TLC of fraction groups of eluted fractions by a chromatography being grouped according to impaction pattern by the TLC.

FIG. 4 is an HPLC chromatogram of chromatographic fraction groups of RgC being grouped according to the result of FIG. 3.

FIG. 5 is a molecular structure diagram of the ginsenoside Rg3 in accordance with an embodiment of the present invention.

FIG. 6 is a series of graphs representing changes in the expression of hChAT (A) and APP (B) in N2a cells transfected with hChAT or APP genes, respectively, when treated with the fraction groups prepared in accordance with an embodiment of the present invention and changes in the expression level of hChAT gene depending on concentration of fraction group No. 3 (C) to be treated.

FIG. 7 is a series of graphs representing changes in the expression level of ChAT (A) and VAChT (B) in N2a cells treated with one of five ginsenosides (Rb1, Re, Rd, Rg1 and Rg3) contained in RgC; changes in the expression level of ChAT (C), VAChT (D) and CHT1 (E) in N2a cells treated with various concentration of Re and Rd resulting in increasing the expression level of ChAT and VAChT; and changes in the amount of acetylcholine in the culture medium of N2a cells (F).

FIG. 8 is a graph showing changes in the expression of MAP2 mRNA of N2a cells treated with one of five ginsenoside (Rb1, Re, Rd, Rg1 and Rg3) at a concentration of 5 μg/ml.

FIG. 9 is a series of fluorescent microscopic images showing immunocytochemistry analyses representing changes in the expression of MAP2 protein (A) and p75 protein (B) in N2a cells treated with NGF, ginsenoside Re and ginsenoside Rd, respectively and an optical microscopic image showing changes in the shape of the cells (C).

FIG. 10 represent a result of western blot analysis representing the expression of ChAT when NGF, ginsenosides Re or Rd with or without K252a, a Trk inhibitor were treated to N2a cells, respectively.

FIG. 11 is a graph showing changes in the expression of ChAT at a level of mRNA when ginsenosides Re, Rd or Re/Rd were treated to N2a cells, respectively.

FIG. 12 is a graph showing a result of quantitative RT-PCR analysis representing the expression of ChAT when ginsenosides Re or Rd with or without K252a, a Trk inhibitor to N2a cells.

FIG. 13 is a graph showing a result of quantitative RT-PCR analysis representing the expression of NGF and NGFR when ginsenosides Re or Rd were treated to N2a cells, respectively.

FIG. 14 is a graph showing a result of quantitative RT-PCR analysis representing the expression of genes related to the degradation of Aβ in HMO6 human microglial cells.

FIG. 15 is a graph showing a change in the expression of caveolin-1, caveolin-2, clathrin, neprilysin (NEP), insulin-degrading enzyme (IDE) according to treating Aβ and/or ginsenoside Rg3 to HMO6 human microglial cells. HMO6 cells were plated on 35 mm cell culture plates or chamber slides and Aβ42 (5 μM) and/or Rg3 (2.5 or 5 μg/ml) were treated for 24 h. Then, the mRNA expression was determined by quantitative RT-PCR. The expression of caveolin (A-B), clathrin (C) which are endocytosis-related genes and NEP (D) and IDE (E) which are genes encoding Aβ-degrading enzymes were compared depending on the presence or absence of Rg3/Aβ42. The results are shown as mean±SD. * P<0.05, ** P<0.01, *** P<0.001.

FIG. 16 is a graph showing representing the expression level of each gene 24 h after treating RgC at various concentrations (10, 25, 50 μg/ml) to HMO6 cells.

FIG. 17 is a result identifying the expression of SRA (scavenger receptor class A) and the absorption of Aβ42 in HMO6.hSRA human microglial cells according to an embodiment of the present invention: A is a series of fluorescence microscopic images of HMO6 human microglial cells transfected with pcDNA3.1(+) vector containing human SRA gene (HMO6.hSRA, right), and non-transfected HMO6 cells (left) taken using a fluorescence microscope; B represents a result of RT-PCR analysis of the expression of SRA in HMO6.hSRA cells; C represents a result of western blot analysis of the expression of SRA in HMO6.hSRA cells; D is a series of fluorescence microscopic images representing immunocytochemical analyses on the uptake of FITC-conjugated Aβ42 in HMO6 (left)/HMO6.hSRA (right) cells after 2 h treating Aβ42. The length of scale bar represents 10 μm.

FIG. 18 is a photograph showing a result of western blot analysis for confirming the degree of phosphorylation of ERK (extracellular-regulated kinase) and p38, according to an embodiment of the present invention. Depending on the presence of Rg3, the levels of phospho-ERK and phospho-p38 protein were investigated by immune-blotting. HMO6 cells were treated with Rg3 (5 μg/ml) after plated on 60 mm cell culture plates and western blot analysis was performed after the cells were placed in phenol red negative/sere-free DMEM medium for 1 h. The western blot analysis was carried out three times independently.

FIG. 19 represents an images showing a result of western blot analysis of the expression of NEP (neprilysin) and IDE (insulin-degrading enzyme) in HMO6 cells (A); graphs representing the above result quantitatively (B and C), and fluorescence microscopic images representing immunocytochemical analyses (D and E). The expression of NEP and IDE in HMO6 cells was analyzed by western blot analysis and immunocytochemistry after treating Aβ42 (5 μM) or Rg3 (2.5 or 5 μg/ml) for 24 h to HMO6 cells in serum-free DMEM medium plated on 60 mm cell culture plates or chamber slides. The results are shown as mean±SD. * P<0.05, ** P<0.01, *** P<0.001.

FIG. 20 is a fluorescence microscopic image showing a result of immunocytochemical analysis investigating the expression of caveolin and clathrin in HMO6 cells treated with or without Rg3. HMO6 cells were treated with Rg3 (5 μg/ml) for 24 h after plated on chamber slides and then the cells were stained with primary antibodies binding specifically to caveolin and clathrin, respectively and counterstained with DAPI. The length of scale bar represents 10 μm.

FIG. 21 is a graph showing a result of the uptake of FITC-labeled Aβ in HMO6 cells. Ctrl represents a control group, the results are shown as ±mean±SD. *** P<0.001.

FIG. 22 is a chromatogram representing a result of HPLC performed to identify ginsenosides from a brain removed 6 h after administrating five ginsenosides (Rb1, Rd, Rd, Rg1 and Rg3) orally to a mouse. The image of the upper left-hand corner in the chromatogram represents a result of RT-PCR analysis reflecting changes in the expression level of ChAT, VAChT and p75 of the removed brain at the level of mRNA.

FIG. 23 is a graph representing a calibrated result of the RT-PCR analysis shown in FIG. 22 using beta-actin as an internal standard, which reflects change in the expression level of ChAT, VAChT and p75 after administrating the five ginsenosides.

FIG. 24 shows a graph represent the expression of SRA and SRB mRNAs in brain tissues after administrating Rg3 to animals according to an embodiment of the present invention (A) and a photographic image representing a protein pattern of western blot analysis on the same brain tissue (B). Ctrl represents a control group, the data are shown as mean±standard deviation (SD). *** P<0.001.

FIG. 25 is a series of graphs representing the observation of behavioral changes after administrating Rg3 to Alzheimer's model mice according to an embodiment of the present invention, A is a graph representing a result of passive avoidance trials (PAT), and B is a graph representing a result of Morris water maze test (MWM). The behaviors were observed upto 4 weeks after administrating Rg3 2 weeks after injecting Aβ42 to the mice. The data are shown as mean±SD. ** P<0.01, *** P<0.001:

: normal control;

◯: Aβ42;

▾: Rg3+Aβ42; and

Δ: Rg3.

### DETAILED DESCRIPTION OF THE INVENTION

In an aspect of the present invention, a method for preparing ginsenoside complex effective for Alzheimer's disease is provided, the method comprises: i) preparing steam-dried unripened ginseng berries by steaming and drying unripened ginseng berries 3 to 10 times; ii) preparing extract of steam-dried ginseng berries by extracting the streamed-dried ginseng berries by extracting the steamed-dried ginseng berries with water, C1 to C4 alcohol or mixture thereof; iii) fractionating the extract of steam-dried ginseng berries by adding solvent comprising ethyl acetate and water to the extract of steam-dried ginseng berries and fractionating to ethyl acetate layer and water layer; and iv) preparing n-butanol fraction by further fractionating the water layer with n-butanol, and forming n-butanol layer thereby and recrystallizing the n-butanol layer with ether.

According to the method, the steam-dried ginseng berries may be prepared by steaming and drying unripened ginseng berries 4 to 9 times.

According to the method, the alcohol may be methanol, ethanol or propanol.

According to the method, the steaming and drying may be carried out for 1-3 hours at a temperature of 80 to 120° C.

According to the method, the ginsenoside complex may comprise at least 2 wt % of ginsenoside Re, at least 8 wt % of ginsenoside Rd and at least 20 wt % of ginsenoside Rg3. The ginsenoside complex may comprise 2 to 7 wt % of ginsenoside Re, 8 to 12 wt % of ginsenoside Rd and 20 to 30 wt % of ginsenoside Rg3. More preferably, the ginsenoside complex may comprise at least 5 wt % of ginsenoside Re, at least 10 wt % of ginsenoside Rd and at least 24 wt % of ginsenoside Rg3.

The method may further comprise isolating fractions corresponding Rf value of 0.4 to 0.7 on thin layer chromatography (TLC) using running solvent of ethyl acetate:methanol=3:1 from eluted fractions of the n-butanol fraction by a chromatography.

According to the method, the chromatography may be a column chromatography using silica gel as stationary phase and mixture of water (A), acetonitrile (B) and chloroform (C) as the mobile phase and the ratio of the components of the solvent may be adjusted by fixing the concentration of C, increasing the concentration of A gradually and decreasing the concentration of B gradually. For example, the ratio of the component of the solvent may be A:B:C=0:80:20 (500 mL), A:B:C=10:70:20 (500 mL), A:B:C=20:60:20 (500 mL), A:B:C=50:30:20 (500 mL), A:B:C=50:30:20 (500 mL), and A:B:C=50:30:20 (500 mL), sequentially.

In another aspect of the present invention, a pharmaceutical composition for treating Alzheimer's disease and improving cognitive function of Alzheimer's patient comprising the ginsenoside complex prepared by the above-described method and at least a pharmaceutically acceptable carriers is provided.

The pharmaceutical composition may be used for treating other diseases involved in amyloid beta deposition such as Lewy body dementia, myositis, and cerebral amyloid angiopathy, considering the action mechanism.

Thus, in another aspect to the present invention, a pharmaceutical composition for treating Lewy body dementia, myositis, or cerebral amyloid angiopathy comprising the ginsenoside complex prepared by the above-described method and at least a pharmaceutically acceptable carriers is provided.

The pharmaceutical composition may be administrated via oral administration or parenteral administration and oral administration is preferred but not limited thereto. When the pharmaceutical composition is administrated via parenteral administration, various routes such as intravenous injection, intranasal inhalation, intramuscular injection, intraperitoneal injection, dermal administration and subcutaneous injection may be used.

In addition, the pharmaceutical composition may be administrated at a dose of 0.1 mg/kg to 1 g/kg, more preferentially at a dose of 1 mg/kg to 500 mg/kg. The dose may be adjusted according to age, sex and body condition of a patient.

The pharmaceutical composition may be formulated in various dosage forms. For examples, the composition may be prepared as a retort pouch, and formulated as powders, pills, and capsules after dried by a drying method such as hot air drying and freeze-drying, jellies prepared by mixing gelation agent such as gelatin. Any known formulations in the art commonly used in the manufacture of pharmaceutical preparations may be used.

Further, at least one pharmaceutically acceptable carrier may be used in the manufacture of the pharmaceutical composition of the present invention. As a carrier, conventional various organic or inorganic carriers, for instance, excipients, lubricants, binders and disintegrating agents in the solid formulation, solvents, solubilizers, suspending agents, isotonic agents, and soothing agents in the liquid formulation may be used. Also, if necessary, additives such as conventional preservatives, antioxidants, coloring agents, sweetening agents, adsorbents, wetting agents, etc. may be suitably used in an appropriate amount.

In another aspect of the present invention, a nutraceutical composition for improving cognitive function of a subject comprising the ginsenoside complex prepared by the above-described method and at least a food additive is provided.

The nutraceutical composition may be formulated as a formulation suitable for nutraceutical compositions such as condense extract, drinks, powders, pills, capsules, tablets (coated tablets, dragees, sublingual tablets, etc.), and jellies.

In still another aspect of the present invention, a method for treating and improving cognitive function of a subject suffering Alzheimer's disease comprising administrating a therapeutically effective amount of ginsenoside complex prepared by the above-described method to a subject suffering Alzheimer's disease is provided.

In another aspect of the present invention, a method for treating Lewy body dementia, myositis, or cerebral amyloid angiopathy comprising administrating a therapeutically effective amount of ginsenoside complex prepared by the above-described method to a subject suffering at least one disease among the above diseases.

Ginsenoside complex containing ginsenoside Rd, Re and Rg3 according to one embodiment of the present invention prepared as described above may be used for treating Alzheimer's disease and for improving cognitive function of Alzheimer's patients since it can pass through the blood-brain barrier (BBB) as well as upregulate the expression of ChAT and AChE related to cognitive function and degrade amyloid beta, a causative substance for neuronal cell death of hippocampus and cerebral cortex.

Terms used in this document are defined as follows:

A “ginsenoside complex (hereinafter referred to RgC)” used in this document mean a fraction prepared by fractionating extract of steam-dried ginseng berries in order to concentrate ginsenosides as major components.

An “active ginsenoside-enriched fraction” used in this document mean a fraction of the RgC whose contents of Rd, Re and Rg3, major effective components are increased by grouping fractions having certain Rf values (0.4 to 0.7) on a TLC plate among fractions eluted from the RgC via a chromatograph.

A “ginsenoside” used in this document mean a type of saponins mainly found in ginseng roots. It exists as various structures such as Rb1, Rb2, Rb3, Rc, Rd, Rg1, Rg2, Rg3, Rg4, by Rg5, Rg6, Rh1, Rh2, Rh3, Rk1, Rk2, Rk3, Re, Rf1, Rf2, and Rf3, etc., depending on extent of attached sugar chains. It has been known that there are changes in the numbers and types of ginsenosides by varying the extent of cleavage of sugar chain from saponin glycosides according to the extent of steaming ginseng roots.

A “choline acetyl transferase (ChAT)” used in this document mean an enzyme to synthesize acetyl choline, a neurotransmitter from choline and it has been known that low level of acetyl choline by the deactivation of ChAT results in decline of cognitive function of a subject.

A “vesicular acetylcholine transporter (VAChT)” used in this document mean a protein which plays a role in the secretion of intracellular acetylcholine to the outside of the cell and the decline of VAChT function also is known as a main causative of memory impairment.

An “amyloid beta (Aβ)” used in this document mean peptides of 36-43 amino acids that are crucially involved in Alzheimer's disease as main component of the amyloid plaques found in the brains of Alzheimer's patients. The peptides result from the amyloid precursor protein (APP), which is being cut by certain enzymes to yield Aβ.

Hereinafter, the present invention will be described in detail by following examples. However, the present invention may be embodied in many different embodiments and is not limited to the embodiments set forth herein, the following examples are provided in order to complete the disclosure of the present invention and inform full scope of the invention to those skilled in the art.

### Example 1

### Preparation of Ginseng Berry Extract

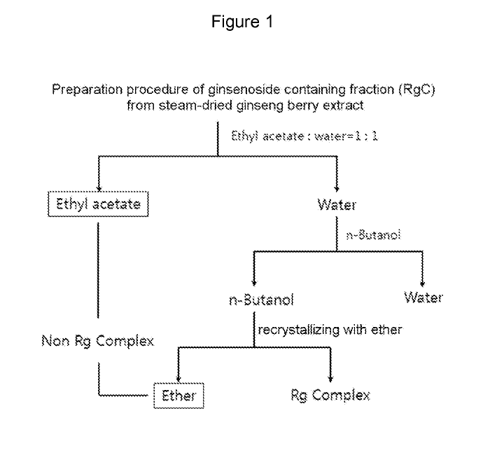

Fresh ginseng (Panax ginseng CA Meyer) berries used in the present were purchased from Korea Genetic Pharm Co. Ltd. (Korea). The immature berries of ginseng used in the present invention just before ripening (unripened berries) is gathered 4 year, the total ginseng berries are steamed for at 100° C. for 2 h and dried at 50° C. for 24 h. These steam and dry procedures were repeated 4 to 9 times, and the final products were prepared after sunlight-drying for 7 days. FIG. 1 is a schematic diagram which illustrates the extraction procedure of the ginsenoside complex (RgC) of steam-dried ginseng berry extract from steam-dried unripened ginseng berries. As shown in FIG. 1 after 100 g of final steam-dried ginseng berries were soaked in 1 L of 80% ethanol at room temperature in a 24-hour period 3 times, repeatedly for three days. And then, the ethanol extract was mixed and evaporated after filtering with a filter paper (Whatman International Ltd., UK). The ethanol extract was fractionated into ethyl acetate layer and water layer with mixture of 1:1 ratio of ethyl acetate and water, the water layer was fractionated into n-butanol layer and water layer with n-butanol and the n-butanol layer was recrystallized with ether and RgC was obtained thereby (FIG. 1). The yield of the RgC is represented by gram of extract per gram of 100 g of dry powder of steam-dried ginseng berries, and about 50% of RgC (13.8 g) was obtained from the ethanol extract (27.5 g).

### Example 2

### Analysis of Ginsenoside Components in RgC

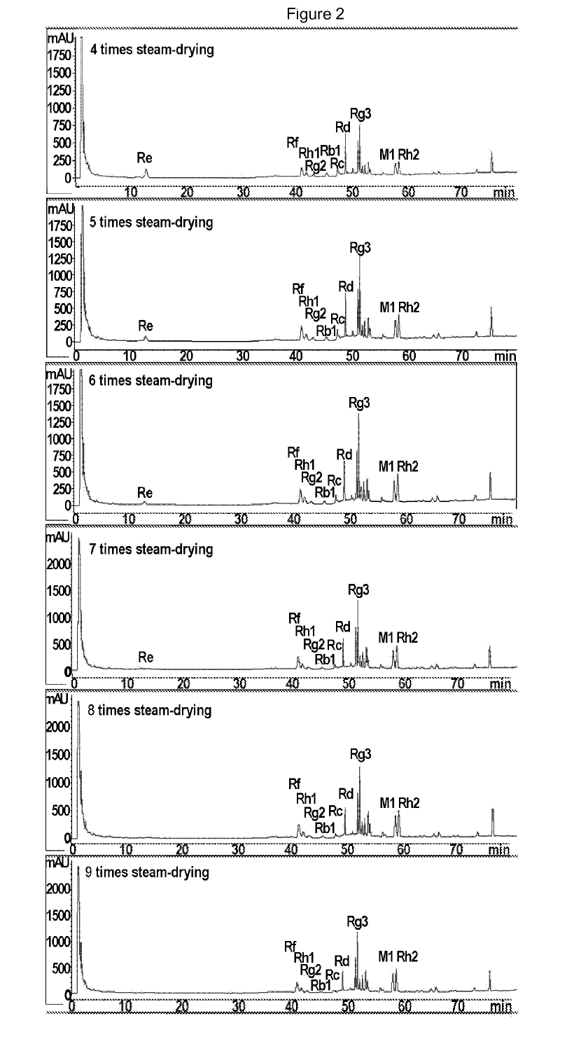

The present inventors performed high-performance liquid chromatography (HPLC 1100 series, Agilent, USA) on the n-butanol fractions obtained from steam-dried ginseng berries 4 to 9 times, in order to identify types and contents of the RgC prepared by the Example 1. Particularly, 10 μl of samples dissolved in distilled water were loaded on a column (Zora SB-Aq C18 column, 4.6×150 mm, 5 μm) and water (A) and acetonitrile (B, HPLC grade, Fisher Scientific, USA) were used as a mobile phase with a flow rate of 1.0 mL/min. The absorbance was analyzed at 203 nm and column temperature was set to 35° C. As a condition of the mobile phase, starting with 79% A, 0-10 min (79-78% A, 21-22% B), 10-11 min (78-77% A, 22-23% B), 11-29 min (77-76% A, 23-24% B), 29-34 min (76-70% A, 24-30% B), 34-44 min (70-68% A, 30-32% B), 44-49 min (68-50% A, 32-50% B), 49-64 min (50-35% A, 50-65% B), 64-78 min (35-0% A, 65-100% B), and 78 to 80 min (0-79% A, 100-21% B) was used and the last was terminated using 300 mL of methanol. In addition, the standard sample (ginsenosides Rg3, ginsenoside Rg1, ginsenoside Re, ginsenoside Rd, and ginsenoside Rb1, BTGin, Korea) to identify the components of RgC was purchased and measured under the same condition. It was confirmed whether the samples exhibit the same retention time as the standard sample and identified each ginsenoside using UV spectra and molecular weight measurements (FIG. 2).

As a result shown in FIG. 2, the major components of RgC were various ginsenoside such as Rg3, Rd, Rh2, Rf, Rb1, Rc, M1, Rh1, Rg2, and Re, and Rg3 is the most abundant, then next was Rd. Types and contents of ginsenosides according to number of steam-drying is summarized in following Table 1.

TABLE 1
Types and contents of ginsenosides in ginsenoside
complex depending on number of steam-drying

| | 4 times | 5 times | 6 times | 7 times | 8 times | 9 times |
|---|---|---|---|---|---|---|
| | steam- | steam- | steam- | steam- | steam- | steam- |
| content (% W/W) | drying | drying | drying | drying | drying | drying |
| Ginsenoside Re | 7.46 | 3.64 | 2.82 | 1.64 | 1.07 | 0.58 |
| Ginsenoside Rf | 7.25 | 8.25 | 7.74 | 9.25 | 9.25 | 9.01 |
| Ginsenoside Rh1 | 3.48 | 3.84 | 3.32 | 4.04 | 3.87 | 3.83 |
| Ginsenoside Rg2 | 2.33 | 2.26 | 1.74 | 1.22 | 0.96 | 1.78 |
| Ginsenoside Rb1 | 2.43 | 2.03 | 1.57 | 1.49 | 1.13 | 0.97 |
| Ginsenoside Rc | 4.62 | 3.54 | 2.85 | 2.41 | 1.82 | 0.94 |
| Ginsenoside Rd | 11.68 | 10.04 | 9.04 | 8.78 | 7.95 | 7.06 |
| Ginsenoside Rg3 | 24.92 | 27.87 | 28.82 | 29.7 | 28.79 | 29.12 |
| Ginsenoside M1 | 8.26 | 8.83 | 9.62 | 9.02 | 9.98 | 10.07 |
| Ginsenoside Rh2 | 8.52 | 9.75 | 10.99 | 10.73 | 11.19 | 12.18 |

As shown in Table 1, it was confirmed that there was difference in the types and contents according to number of steam-drying of ginseng berries by the HPLC analysis and total ginsenoside content in 7 times steam-drying group was the highest. Interestingly, as number of steam-drying increase the contents of Rf, Rh1, Rg3, and M1 were observed to increase and Rh2, Re, Rc, Rd were shown to have the highest content in 4 times steam-drying group and then decreased according to increase number of steam-drying. Thus, from the result the present inventors thought that mixing RgC of 4 times steam-drying whose main contents are 5 types of ginsenosides (Re, Rg2, Rb1, Rc, and Rd) and which includes Re, Rd, and Rg3 which are expected as being related to brain among Re, Rf, Rd and Rg3 showing the highest content on HPLC analysis and Rg1 and Rb1 which were confirmed to correlate with the central nervous system in previous studies and RgC of 7 times steam-drying showing the highest total ginsenoside content provides ideal condition for producing useful ginsenosides. Thus, the present inventors mixed the RgC of 4 times steam-drying and the RgC of 7 steam-drying with a ratio of 50:50 and used it following in vitro experiments. In addition, the present inventors performed various experiment to identify effective ginsenosides for improving cognitive function and degrading Aβ in the brain of Alzheimer's patents.

### Example 3

### The Concentration of the Active Ingredient Through a Chromatography of RgC

The present inventors grouped RgCs from 4 time/7 time steam-drying ginseng berries which are the optimum number of steam-drying. The grouping was performed by using the column chromatography according to the method of the above Example 2, elutes were obtained using 45 mL each of 74 test tubes, and TLC was used for grouping each test tube.

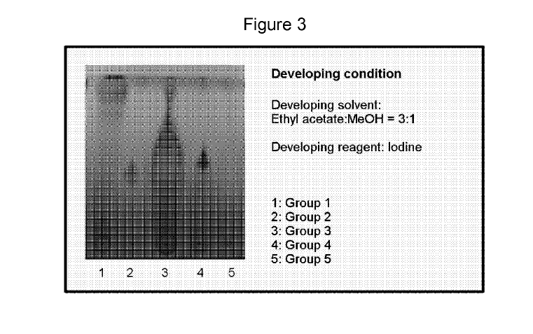

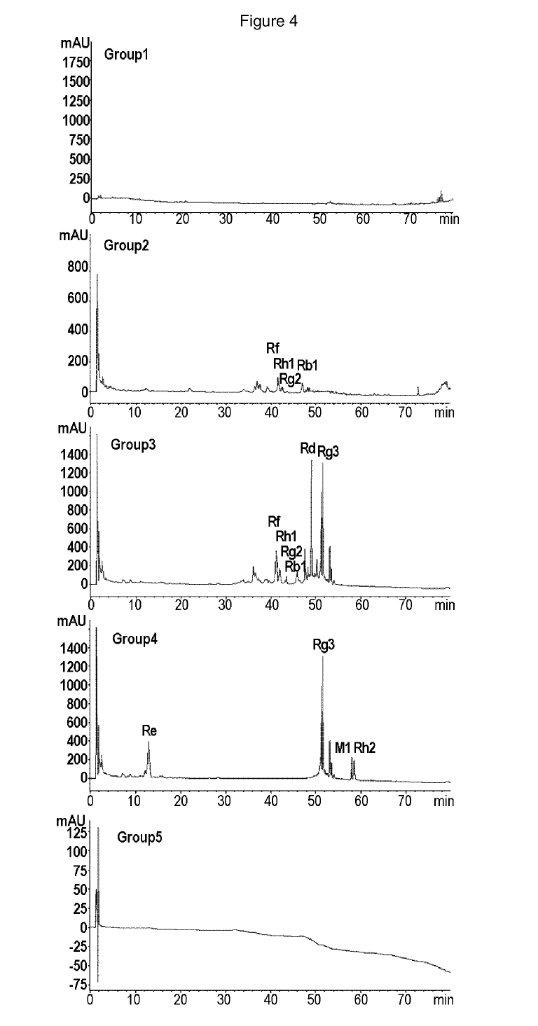

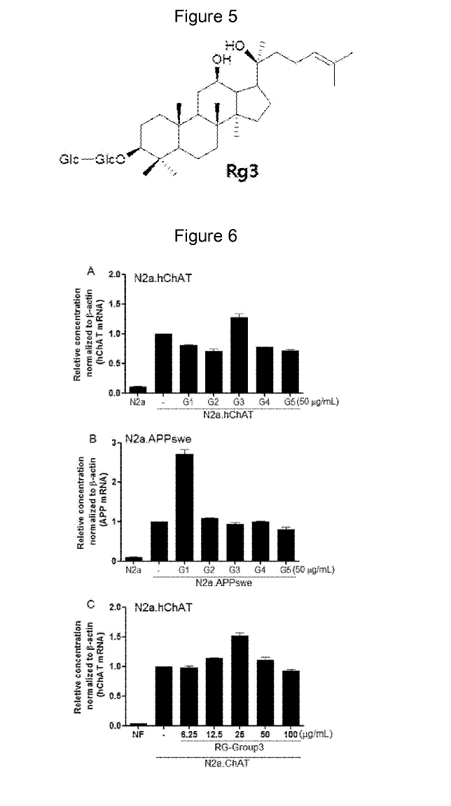

That is, while monitoring the TLC for each test tube, tubes with changed Rf value were designated as new group. Group 1 was collected as the first part in the column chromatography, group 2 corresponded to Rf value of about 0.5, group 3 showed various smeared spots upto Rf value of 0.48, and group 4 corresponded to Rf value of about 0.6 which was not shown in group 3. Group 5 was the last part of the HPLC (water:acetonitrile:chloroform=50:30:20, 500 ml to 300 ml of methanol) (FIG. 3). HPLC results for each group are as shown in FIG. 4. As a result, while the group 2 included a small amount of ginsenosides, the group 3 included large amount of Rf, Rd, and Rg3 (FIG. 5) and group 4 included Re, Rg3, M1 and Rh1, and particularly, the content of Re was higher than RgC.

### Example 4

### Cognitive Function Improvement Tests

4-1: Investigation of Cognitive Function Improvement of RgC

The present inventors analyzed effect of ginsenoside fraction groups 1 to 5 isolated in the Example 3 on the expression of ChAT related to cognitive function in the CNS and amyloid precursor protein (APP) known to be related to pathology of Alzheimer's disease in order to investigate whether RgC is capable of improving cognitive function of a subject suffering Alzheimer's disease actually.

The present inventor reported that ChAT overexpressing human neural stem cells restored cognition with an increase of ACh levels in rat model (Park et al., Exp. Neurol., 234: 521-526, 2012). This implies that the increase of the ChAT level in cholinergic neurons plays a critical role in cognition enhancement and thus the cholinergic gene locus “VAChT/ChAT” can be a putative target for therapeutic approaches on AD. Thus the present inventor prepared transfected Neuro-2a (N2a) cells that over-express the human ChAT and APP (designated as “N2a.hChAT” and “N2a.hAPPswe”, respectively, FIG. 6) and the N2a.hChAT and N2a.hAPPswe cells were treated with the faction groups 1 to 5 isolated in the Example 3 at a centration of 50 μg/ml. Neuro-2a mouse neuroblastoma cells (Korean Cell Line Bank, Korea) were grown in DMEM supplemented with 10% fetal bovine serum (FBS), 100 U/m penicillin, and 100 μg/ml streptomycin (Invitrogen, USA) under 5% CO₂ at 37° C. in tissue culture flasks. For all experiments, cells were grown to greater than 90% confluency and subjected to no more than 20 cell passages. The medium was changed every 2-3 days. Subconfluent cells were harvested and seeded at a density of 5×10⁵ cells or 5×10⁶ cells in poly-L-lysine-coated 35 mm or 60 mm culture plates. To recover the cells that are not completely saturated for analysis, poly-L-lysine coated 35 mm or 60 mm culture was seeded at a concentration of 5×10⁵ or 5×10⁶ cells on the plates. The extent of expression of hChAT and APP in the N2a.hChAT and N2a.hAAPswe cells treated with the faction groups 1 to 5 was quantified with RT-PCR using primers described in the below Table 2. At this time, beta-actin was used as an internal control.

Specifically, total RNA extracts from N2a cells were prepared using the Trizol method (Invitrogen, USA) and cDNA was synthesized from RNA by reverse transcription of 1 μg of total RNA using a reverse transcription system (Improm-II, Promega, USA) and oligo dT primers in a total volume of 20 μL. PCR amplification was performed using the primers described in Table 2 (Bioneer, Korea). Quantitative real-time PCR reactions were run on a Rotor-Gene 6000 (Corbett Research, Australia) using SensiMix™ SYBR Hi-ROX Kit (Bioline, U.K.) in 20 μL reaction mixtures. Each real-time-PCR master mix contained 10 μL 2× enzyme master mix, 7.0 μL RNase free water, 1 μL of each primer (10 pM each), and 1 μL diluted template. The PCR was performed with an initial pre-incubation step for 15 min at 95° C., followed by 45 cycles of 95° C. for 15 s, annealing at 52° C. for 15 s, and extension at 72° C. for 10 s. Melting curve analysis was used to confirm formation of the expected PCR product, and products from all assays were additionally tested with 1.2% agarose gel electrophoresis to confirm the correct lengths. An inter-run calibrator was used, and a standard curve was created for each gene to obtain PCR efficiencies. Relative sample expression levels were calculated using Rotor-Gene 6000 Series Software 1.7, and were expressed relative to β-actin and corrected for between-run variability. Data for the experimental samples were expressed as a fold unit the internal control gene.

TABLE 2
Gene and primers used for RT-PCR

| | | Nucleotide sequences | Product | GenBank |
|---|---|---|---|---|
| Gene | Primer | (SEQ ID Nos.) | size (bp) | Accession No. |
| ChAT | ChAT-F | 5′-gttccccagaaactcaaggc-3′ (1) | 280 | NM_009891 |
| | ChAT-R | 5′-gagtggccgatctgatgttg-3′ (2) | | |
| VAChT | VAChT-F | 5′-ttgatcgcatgagctacgac-3′ (3) | 246 | NM_021712 |
| | VAChT-R | 5′-ccactaggcttccaaagctg-3′ (4) | | |
| APP | APP-F | 5′-gtggtccgagttcccacgac-3′ (5) | 515 | Y00264.1 |
| | APP-R | 5′-tcataaccggggaccggatc-3′ (6) | | |
| MAP2 | MAP2-F | 5′-accacacctgcagtggagaa-3′ (7) | 227 | M21041 |
| | MAP2-R | 5′-aatctggacctggttcctgc-3′ (8) | | |
| P21 | p21-F | S′-agtgtgccgttgtctcttcg-3′ (9) | 152 | AB017817 |
| | p21-F | 5′-tcaaagttccaccgttctcg-3′ (10) | | |
| TrkA | TrkA-F | 5′-ccagatgcccaatgactctg-3′ (11) | 215 | NM_001033124 |
| | TrkA-R | 5′-cccagcacgtcacattcttc-3′ (12) | | |
| NGF | NGF-F | 5′-tactgcaccaatagctgccc-3′ (13) | 200 | M35075 |
| | NGF-F | 5′-tccggtgagtcctgttgaaa-3′ (14) | | |
| NGFR (p75) | NGFR-F | 5′-aagagatccctggtcgatgg-3′ (15) | 226 | NM_012610 |
| | NGFR-F | 5′-gcagccaagatggagcaata-3′ (16) | | |
| CHT1 | CHT1-F | 5′-tctgcagtacctctgccctg-3′ (17) | 213 | AJ401467 |
| | CHT1-F | 5′-gctccgaacacaagcacagt-3′ (18) | | |
| human SRA | hSRA-F | 5′-ggagatcgaggtcccactg-3′ (19) | 197 | NM_138715 |
| | hSRA-R | 5′-tgtttccactccccttttcc-3′ (20) | | |
| human SRB | hSRB-F | 5′-accctaaccaggaggcacac-3′ (21) | 212 | NM_005505 |
| | hSRB-R | 5′-gtgaagagtctccccctcca-3′ (22) | | |
| mouse SRA | mSRA-F | 5′-ttcactggatgcaatctcca-3′ (23) | 195 | AF203781 |
| | mSRA-R | S′-acgtgcgcttgttcttcttt-3′ (24) | | |
| mouse SRB | mSRB-F | 5′-acaaatggaacggactcagc-3′ (25) | 202 | NM_016741 |
| | mSRB-R | 5′-gtgaagagtctccccctcca-3′ (26) | | |
| NEP | NEP-F | 5′-atcagcctctcggtccttgt-3′ (27) | 241 | NM_000902 |
| | NEP-R | 5′-tggaagacagcgcaagactc-3′ (28) | | |
| IDE | IDE-F | 5′-cagtgatcccaccacggata-3′ (29) | 264 | NM_004969 |
| | IDE-R | 5′-gtgccctagacaggtttgca-3′ (30) | | |
| MMP9 | MMP9-F | 5′-ctcctactctgcctgcacca-3′ (31) | 257 | NM_004994 |
| | MMP9-R | 5′-aactacgaccgggacaagct-3′ (32) | | |
| Caveolin1 | Caveolin1-F | 5′-cagacgagctgagcgagaag-3′ (33) | 190 | BT007143 |
| | Caveolin1-R | 5′-gacagcaagcggtaaaacca-3′ (34) | | |
| Caveolin2 | Caveolin2-F | 5′-agccatgccctctttgaaat-3′ (35) | 239 | BT007051 |
| | Caveolin2-R | 5′-acgctcgtacacaatggagc-3′ (36) | | |
| Clathrin | Clathrin-F | 5′-aagcccttgatgccaattct-3′ (37) | 244 | BT007170 |
| | Clathrin-R | 5′-agatcgttcgtccggtttct-3′ (38) | | |
| GAPDH | GAPDH-F | 5′-ggagccaaaagggtcatcat-3′ (39) | 202 | NM_002046 |
| | GAPDH-R | 5′-accacagtccatgccatcac-3′ (40) | | |
| β-actin | P-actin-F | 5′-tacagcttcaccaccacagc-3′ (41) | 187 | NM_007393 |
| | P-actin-R | 5′-aaggaaggctggaaaagagc-3′ (42) | | |

As a result shown in FIG. 6, the expression of hChAT gene in group 3 which is a primary screened substance was increased significant (A) but the expression of APP gene was not decreased at the same dose of 50 μg/mL (B). In addition, when the group 3 was treated to cells by varying the concentration, it was observed increased expression in a dose-dependent manner upto 25 μg/mL (C). From the results, it was confirmed that the RgC according to an embodiment of the present invention could be a therapeutic candidate for improving cognitive function of Alzheimer's patient.

4-2: Determination of Effective Components

Thus, the present inventors carried out experiments on five main ginsenosides contained in the RgC (Rb1, Rd, Re, Rg1 and Rg3) similar with those of Example 4-1, in order to verify which substances within the RgC have the cognitive improvement.

4-2-1: ChAT, VAChT and CHT1 Expression Regulation

In order to examine whether five ginsenosides affect the expression of ChAT related to the synthesis of acetylcholine and VAChT controlling extracellular secretion of acetylcholine, the present inventor analyzed the expression level of ChAT and VAChT after treating the five ginsenosides to N2a glioblastoma cells, respectively.

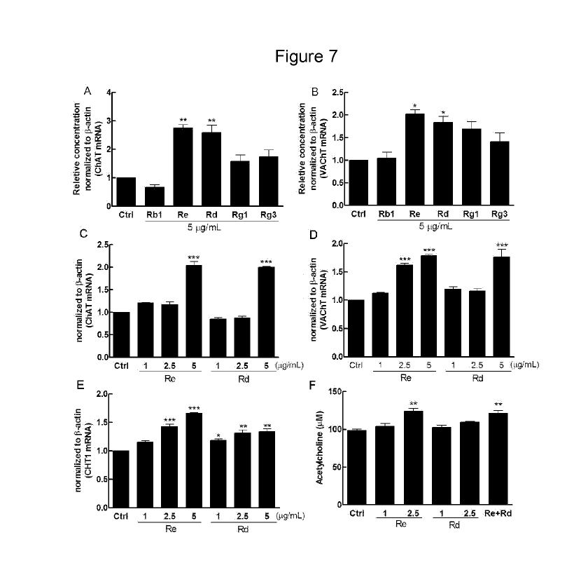

As a result, as shown in FIG. 7, the expression of ChAT and VAChT mRNA in N2a cells was significantly increased in case of treatment with Re and Rd, while other ginsenosides (Rb1, Rg1, and Rg3) showed no significant increase (FIGS. 7A and 7B). In addition, the ginsenoside Rd and Re resulted in increased expression of ChAT and VAChT in a dose-dependent manner (FIGS. 7C and 7D). Although Rb1 has been reported to facilitate the release of acetylcholine (ACh) from hippocampal slices in a previous study (Benishin et al., Pharmacol., 42: 223-229, 1991), the present invention produced a negative result.

In addition, the present inventor investigated change in the expression level of CHT1, high-affinity choline transporter controlling uptake of choline with a similar method described above. As a result, the expression of CHT1 was also increased by the treatment of ginsenosides Re and Rd (FIG. 7E).

Further, the present inventor measured contents of acetylcholine in culture medium of N2a cells treated with Rd and/or Rd (1, 2.5 and 5 μg/ml) for 48 h using a commercial acetylcholine assay kit (Cell Biolabs, USA), based on the enzyme driven reaction that will detect acetylcholine via acetylcholine esterase enzyme and choline oxidase according to the manufacturer's instruction (FIG. 7F). As shown in FIG. 7F, acetylcholine production was increased significantly with ginsenoside Rd, Re or Rd/Re mixture, and this result suggest that increase of expression level of acetylcholine-synthesizing gene leads to increase of the biosynthesis of acetylcholine actually.

4-2-2: Expression Regulation of MAP-2 and p75

In addition, the present inventor investigated changes in the expression of MAP-2 in N2a cells according to the treatment of ginsenosides, in view of the point that ChAT is an important enzyme involved in producing neurotransmitter acetylcholine and a marker for the differentiation of cholinergic neurons, assuming that general neuron markers (e.g. MAP-2) and the earliest cholinergic surface markers (e.g., p75, which is an NGF receptor) should be expressed together with cholinergic cell markers such as the enzyme ChAT and VAChT.

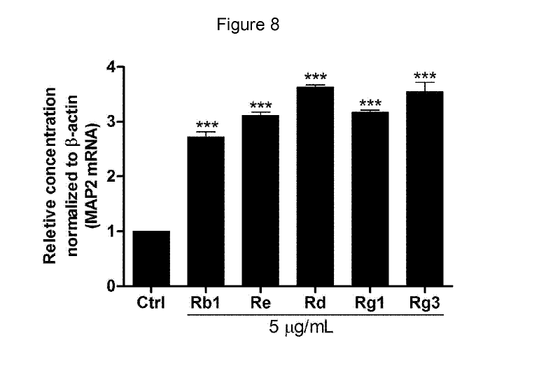

Upon carrying out real-time RT-PCR by a method described in the Example 4-2-1, as shown in FIG. 8, the expression of MAP-2 was significantly increased in N2a cells treated with ginsenoside Rd, and Re. Interestingly, the other three ginsenosides which did not induce the expression of ChAT and VAChT also induced the expression of MAP-2.

Followed by the RT-PCR analysis, the present inventor investigated whether ginsenosides Re and Rd induce the expression of MAP-2 and p75 and the differentiation of neurons actually, via immunocytochemistry and morphological analysis using optical microscopic observation.

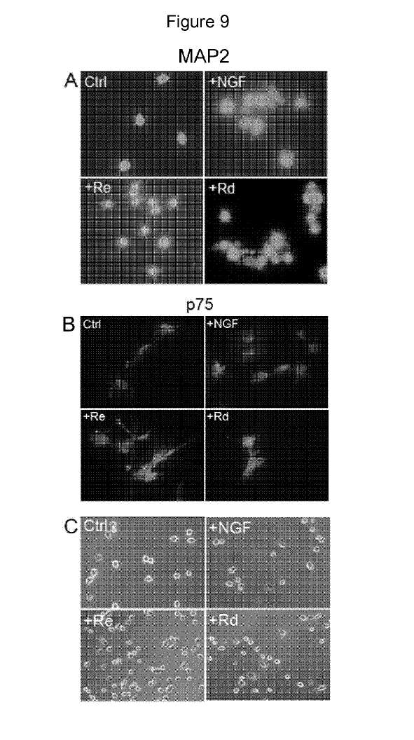

Specifically, Cultured N2a cells were fixed in 4% paraformaldehyde (in PBS) for 15 min, washed twice with PBS supplemented with 100 mM glycine for 5 min, and incubated with permeabilization buffer consisting of 0.1% Triton X-100 (Sigma-Aldrich, USA) in PBS for 30 min at room temperature. Blocking was performed with 1% bovine serum albumin (BSA, Sigma-Aldrich, USA) for 30 min at room temperature. Then Microtubule-Associated Protein-2 (MAP-2) or VAChT mouse monoclonal antibody 1:200 (Santa Cruz Biotechnology, USA) was added to 1% BSA in PBS with Tween 20 (PBST) and incubated for 2 h at room temperature. Cells were then washed three times with PBS before fluorescein isothiocyanate (FITC)-conjugated anti-mouse immunoglobulin G (IgG) 1:200 (Cell Signaling Technology, USA) was added to 1% BSA for 1 h at room temperature. The cells were rinsed and counterstained with 4,6-diamidino-2-phenylindole (DAPI, Sigma-Aldrich) for 10 min, followed by two PBS washes. Cultures were visualized with an inverted fluorescent microscope system (Eclipse Ti-S; Nikon, Japan) at a magnification of ×600 and optical inverted microscopy (OLYMPUS CKX41, Japan) at a magnification of ×200 (FIG. 9).

As a result, as shown in FIG. 9, ginsenosides Re and Rd stimulated effectively the production of MAP-2 protein and switched to p75-positive N2a cells to the same or more extent as NGF (FIGS. 9A and 9B). FIG. 9C shows the morphological changes of N2a cells induced by NGF, Re, and Rd. In case N2a cells were cultured without any treatment, the shape was mostly rounded; whereas cells treated with NGF, Re, and Rd induced the production of distinctive multiple projections (club shapes).

4-2-3: ERK and Akt Signaling Regulation

The cholinergic marker ChAT and VAChT are known activate the ERK and Akt lower level path for cholinergic gene expression (Williams et al., Neurosci. Lett., 413: 110-114, 2007; Madziar et al., J. Neurochem., 107: 1284-1293, 2007) by controlling the NGF-TrkA signaling pathway. Thus, the present inventor investigated whether ginsenosides Rd and Re regulate activity of ERK and Akt in N2a cells using through western blot analysis. Specifically, N2a cells were lysed in 1% radioimmunoprecipitation assay (RIPA) buffer containing protease and phosphatase inhibitors (Roche, Mannheim, Germany) and whole cell lysates were separated by 10% sodium dodecyl sulfate polyacrylamide gel electrophoresis (SDS-PAGE). After electrophoresis, proteins were transferred onto polyvinylidene fluoride (PVDF) membranes and the membranes were blocked with 5% skim milk in Tris-buffered saline solution containing 0.1% Tween-20. The membrane was then immunoblotted with primary antibodies, anti-phospho-Akt, anti-phospho-extracellular signal-regulated protein kinase (ERK), anti-ChAT and anti-f3-actin (Santa Cruz Biotechnology, Santa Cruz, USA), and this was followed by incubation with horseradish peroxidase-conjugated anti-rabbit or anti-mouse secondary antibodies (Stressgen, USA). Blots were developed using an enhanced chemiluminescence (ECL) solution (Thermo, USA).

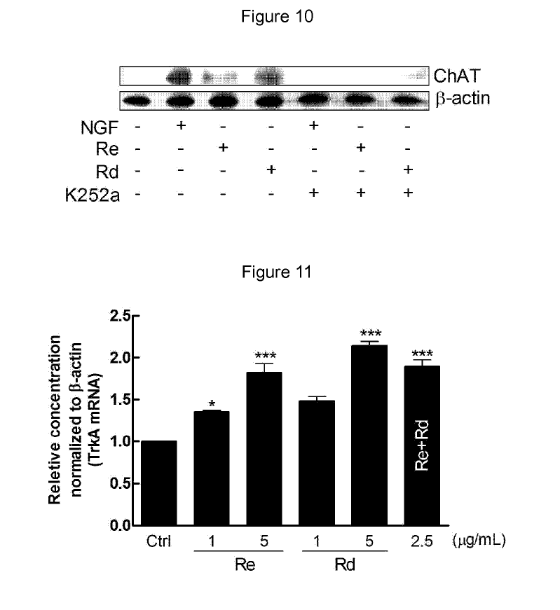

As a result, as shown in FIG. 10, Re and Rd could activate ERK and Akt with phosphorylation as much as NGF, suggesting that Re and Rd are closely related with the NGF-TrkA signaling pathway (FIG. 10). This activation was coincidentally observed with co-treatment with Re and Rd.

On the other hand, the present inventor investigates the expression level of TrkA gene after treating ginsenoside Rd (5 μg/ml), Re (5 μg/ml) or mixture of Rd/Re (each 2.5 μg/ml) to N2a cells for 24 h, using quantitative RT-PCR method, similar to the method described in the Example 4 (FIG. 11).

As a result, as shown in FIG. 11, it was confirmed that ginsenoside Rd, Re and mixtures thereof increased the expression of TrkA in a dose-dependent manner.

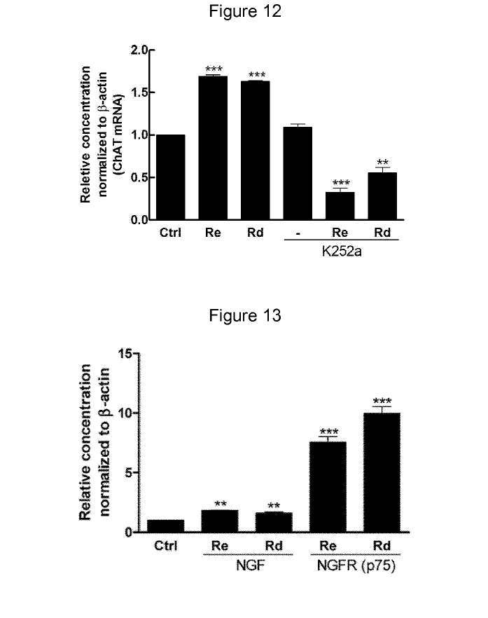

Since NGF-Trk signal pathway upregulates ChAT and VAChT, it is important to know whether ginsenosides Rd and Re regulate Trk activity directly or at least indirectly. Therefore, the inventor investigated whether the expression of ChAT and VAChT was blocked by treating K252a, an inhibitor against protein kinase C (PKC) and Trk together with ginsenoside Rd or Re, in order to determine more closely whether the expression of cholinergic surface markers and the differentiation of neurons by the treatment of ginsenosides Rd and Re are caused whereby they act NGF-TrkA pathway directly. Particularly, N2a cells were treated with or without K252a (50 nM) in the presence of NGF (100 ng/mL), Re (5 μg/mL) or Rd (5 μg/mL) for 48 h. Total RNA and protein lysates were isolated as described in the Example 4-1 (FIG. 12). As a result, as shown in FIG. 12, ChAT expression in N2a cells was inhibited in case of co-treatment with K252a+ Re or +Rd compared with Re and Rd single treatments, suggesting that Re and Rd are related to the Trk signaling pathway and mRNA expression under the same conditions. In addition, the present inventor performed quantitative RT-PCR in order to verify whether the treatment of ginsenosides Rd and Re affect the expression of NGF and NGFR in N2a cells. As shown in FIG. 13, it was confirmed that NGF and NGFR (p75) mRNA levels were significantly increased when treated with Re and Rd.

The result suggests that ginsenosides Rd and Re in accordance with an embodiment of the present invention, main components of RgC have effective activity on increasing acetylcholine, a neurotransmitter through an NGF-like signal pathway.

### Example 5

### Analysis of Aβ Plaque Degrading Activity

Although Rg3 among main components of RgC has been identified not to improve cognitive function, Aβ42 degrading activity of Rg3 was investigated, in order to verify whether it can suppress the death of brain cells due to the accumulation of Aβ42, which is a main symptom of Alzheimer's disease as follows.

5-1: Reagents

Ginsenoside Rg3, which is derived from Panax ginseng, with purity of at least 98%, was purchased from BTGin (Korea) (FIG. 5). A concentrated stock solution (10 mg/ml) of Rg3 was prepared by dissolving it in 10% dimethylsulfoxide (DMSO; Sigma-Aldrich, St. Louis, Mo., USA). The solution was kept at 4° C. until use. The stock solution was further diluted 1:10 in culture media for cell treatments. The final DMSO concentration of in the culture media (<0.25%) had no effect on microglial activation (data not shown). Lyophilized Aβ42 (Calbiochem, San Diego, Calif., USA) was diluted to 5 mg/ml in distilled water and stored at −20° C. until use.

5-2: Cell Culture

HMO6 human microglial cell line was kindly provided by Dr. SU Kim (Chung-Ang University, Korea). HMO6 cells were grown in Dulbecco's Modified Eagle's Medium (DMEM) (Hyclone, USA) supplemented with 10% fetal bovine serum, 100 U/ml penicillin, and 100 μg/ml streptomycin (Invitrogen, USA). Cultures were maintained under 5% CO₂ at 37° C. in tissue culture flasks. Cells were grown to >90% confluency and subjected to no more than 20 cell passages. Medium was changed every 2-3 days. Subconfluent cells were harvested and seeded at a density of 5×10⁵ cells or 5×10⁶ cells in poly-L-lysine-coated 35-mm or 60-mm culture plates. After 24 h of plating, the medium was replaced with serum-free DMEM, washed once with phosphate buffered saline (PBS), and treated with Rg3 or Aβ42.

5-3: Animals

Male BALB/c mice (10 weeks old) were purchased from Samtaco (Korea), and were adapted to laboratory conditions (temperature: 20±2° C., relative humidity: 50%, light/dark cycle: 12 h) for 1 week. Five-week-old male Sprague-Dawley rats were obtained from Daehan-Biolink (Korea) for learning and memory testing. The animals (n=5/group) were maintained at a constant temperature (23±2° C.), relative humidity, 55±10%, and 12-h light/dark cycle. The animals were fed a standard rodent chow and purified water ad libitum. Lyophilized Aβ42 (0.5 mg, Bachem Torrance, USA) was resuspended in PBS (pH 7.4) by shaking for 7 days at room temperature to prepare the fibrillar-aggregated Aβ42 (fAβ42). At 10 weeks of age, mice were anesthetized, and 1 μl fAβ42 (350 μM stock concentration) was stereotaxically injected unilaterally into the parietal cortex (anterior 1.7 mm, lateral 1 mm, and ventral 1 mm to the bregma above the hippocampus within 1 min) (Mohajeri et al., J. Biol. Chem., 277: 35460-35465, 2002)

The animal experiments were approved by the Gangneung-Wonju National University Animal Care and Use Committee (Approval No. GWNU-2013-23), and all procedures were conducted in accordance with the Guide for Care and Use of Laboratory Animals published by the U.S. National Institutes of Health.

5-4: Quantitative Real-Time Polymerase Chain Reaction

Total RNA extracts from HMO6 cells or perfused brain tissue were prepared using the Trizol method (Invitrogen). cDNA was synthesized from RNA by reverse transcription of 1 μg total RNA using the Improm-II reverse transcription system (Promega, Wis., USA) and oligo dT primers in a total volume of 20 μl. PCR amplification was performed using the primers listed in Table 1 (Bioneer, Korea). qPCR reactions were run on a Rotor-Gene 6000 (Corbett Research, Sydney, Australia) using a SensiMix™ SYBR Hi-ROX Kit (Bioline, UK) in 20 μl reaction mixtures. Each real-time-PCR master mix contained 10 μl 2× enzyme master mix, 7 μl RNase free water, 1 μl of each primer (10 pM each), and 1 μl diluted template. The PCR was performed with an initial pre-incubation step for 15 min at 95° C., followed by 45 cycles of 95° C. for 15 s, annealing at 52° C. for 15 s, and extension at 72° C. for 10 s. Melting curve analysis was used to confirm formation of the expected PCR product, and products from all assays were additionally tested on 1.2% agarose gel electrophoresis to confirm the correct lengths. An inter-run calibrator was used, and a standard curve was created for each gene to obtain PCR efficiencies. Relative sample expression levels were calculated using Rotor-Gene 6000 Series Software 1.7 and were expressed relative to β-actin for mice or glyceraldehyde 3-phosphate dehydrogenase for HMO6 cells and corrected for between-run variability. Data for the experimental samples are expressed as a percentage of the internal control gene. Primers were used as described in Table 1.

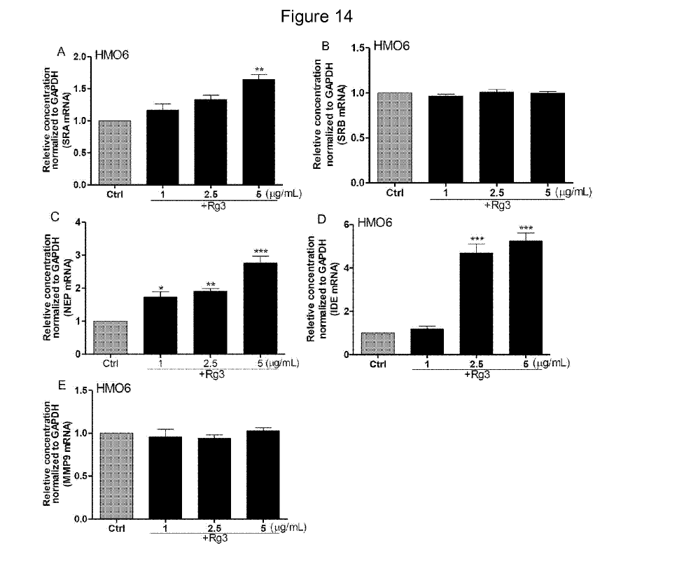

At first, the present inventor examined whether Rg3 influenced expression of ADE, such as NEP, IDE, and matrix metalloproteinase-9. As a result shown in FIG. 14, Rg3-treated HMO6 cells dose-dependently increased SRA, NEP, and IDE mRNA expression (FIG. 14A-E). Notably, the zinc-metalloprotease IDE was expressed about six-fold higher than that in the untreated control group, indicating that degradation of Aβ peptides within the cell would be highly activated. In addition, IDE and NEP were distinctively expressed in cells after treatment with 5 μg/ml Rg3.

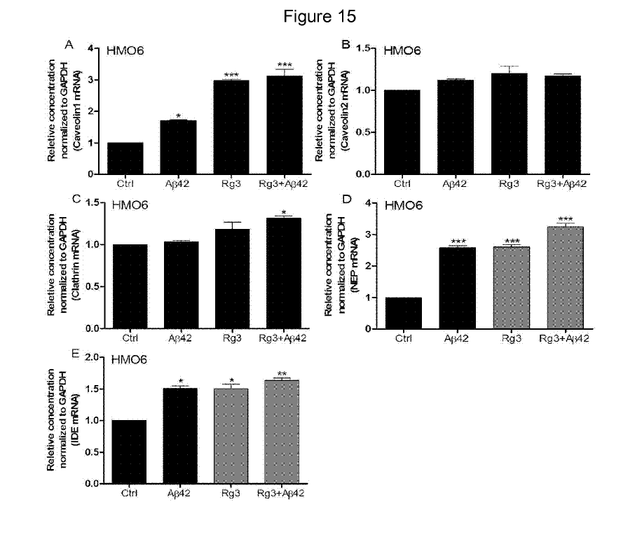

Endocytosis of membrane proteins and lipids, extracellular ligands, and soluble molecules from the cell surface into the internal membrane compartment is a hallmark of all eukaryotic cells and important for various cellular signaling events. Thus, we investigated if Rg3 regulates two cellular proteins, clathrin and caveolin, which control endocytosis of Aβ peptides by binding with SRA (Alarcón, R. et al., J. Biol. Chem., 280: 30406-30415, 2005). FIG. 15A to 15C shows that Rg3 promoted Aβ42 uptake into HMO6 cells via a caveolin1 and clathrin-mediated endocytic mechanism. Coincidentally, NEP and IDE mRNA also increased significantly, suggesting effective Aβ42 degradation after internalization. In general, both clathrin-mediated endocytosis and an alternative internalization pathway through caveolae have been linked to activation of mitogen-activated protein kinases (MAPKs). Therefore, the present inventor assessed whether Rg3 activated the MAPK signal pathways such as p38 and ERK, which accelerate endocytosis and regulate endosomal maturation and cargo degradation, respectively (Sorkin, A. and Zastrow, M., Nat. Rev. Mol. Cell Biol., 10: 609-622, 2009).

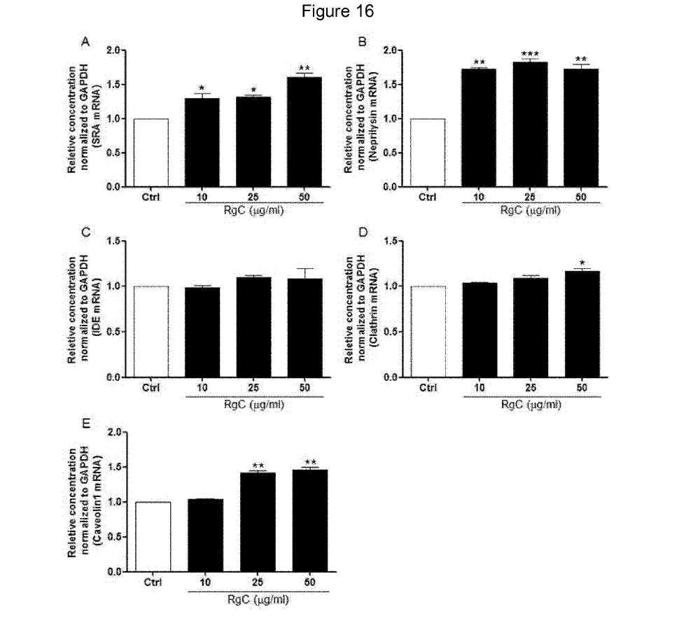

On the other hand, the present inventor analyzed the expression level of SRA, neprilysin, IDE, clathrin and caveolin 1 after treating 0 to 50 μg/ml of RgC to HMO6 cells in order to investigate whether RgC containing Rg3 as a main component has similar activity with the Rg3. As a result shown in FIG. 16, SRA showed dose-dependent increased expression pattern (FIG. 16A), neprilysin was increased although there was no dose-dependent pattern within the range of concentrations treated (FIG. 16B), clathrin (16B) and caveolin 1 (16E) showed dose-dependent increased expression pattern although the extents were somewhat low. However, there was no significant difference in case of IDE (16C). Nonetheless, since the expression pattern of RgC treating groups was similar with that of Rg3 treating groups, it was confirmed that RgC can induced the expression of gene involved in removing Aβ42.

5-5: Construction of the Expression System and Transfection of Cells hSRA to HMO6 Cell

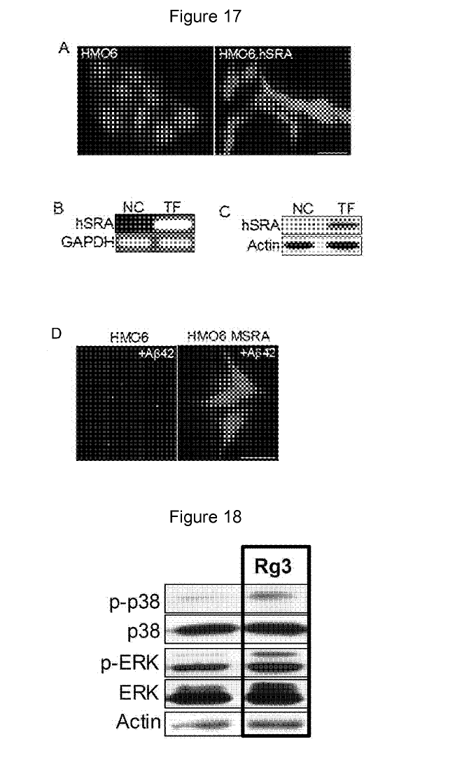

Full length human SRA cDNA (Accession NM_{—}138715) was obtained by PCR from human small intestine Marathon-Ready cDNA (Clontech, USA) through the PCR. The PCR product was inserted to a plasmid pGEM-T easy vector (Promega, USA), the integrity of inserted cDNA was confirmed by DNA sequencing through ABI 3100 DNA sequencer (Applied Biosystems, USA). After cutting the plasmid vector with a restriction enzyme EcoRI-XhoI, hSRA cDNA was inserted into the pcDNA3.1(+) mammalian expression vector (Invitrogen, USA). Then, the pcDNA3.1(+) containing the full-length hSRA cDNA was subcloned in E. coli (DH5a), purified using a plasmid DNA miniprep kit (QIAprep Spin Miniprep Kit, Qiagen, USA). The day before transfection, HMO6 cells were trypsinized, counted, and seeded on 35-mm culture plates at the appropriate density (4×10⁵ cells/ml). When cells were 80% confluent, the culture medium was changed to serum-free DMEM, and the pcDNA3.1(+).hSRA was transfected to the HMO6 cells using PolyFect® Transfection Reagent kit (Qiagen, USA) and cultured for 24 h. G418 (500 μg/ml) (Promega, USA), an inhibitor of cell elongation, was treated for 4 weeks until positive clones were selected. Successful transfection was confirmed by conventional PCR (FIG. 17A to 17C).

As a result shown in FIG. 17, it was confirmed that HM06.hSRA was over-expressed normally.

In addition, the present inventor analyzed Aβ42 uptake using FITC-conjugated Aβ42 in order to verify whether hSRA induces Aβ42 uptake. As a result shown in FIG. 17D, the HMO6.hSRA cells showed remarkable uptake of FITC-conjugated Aβ42 within 2 h when compared to that of the normal control.

5-6: Western Blot Analysis

HMO6 cells were lysed in 1% RIPA buffer containing protease and phosphatase inhibitors (Roche, Mannheim, Germany) and whole-cell lysates were separated on 10% sodium dodecyl sulfate-polyacrylamide gel electrophoresis. After electrophoresis, the proteins were transferred to polyvinylidene fluoride membranes, and the membranes were blocked with 5% skim milk in Tris-buffered saline solution containing 0.1% Tween-20. The membranes were then immunoblotted with primary antibodies, anti-phospho-p38, anti-p38, anti-phospho-ERK, anti-ERK, and anti-f3-actin (Santa Cruz Biotechnology, USA), followed by incubation with horseradish peroxidase-conjugated anti-rabbit or anti-mouse secondary antibodies (Stressgen, USA). Blots were developed using an enhanced chemiluminescent solution (Thermo, USA).

As shown in FIG. 18, Rg3 promoted the phosphorylation of p38 and ERK in HOME microglial cells. These results suggests that Rg3 contributes to receptor-mediated Aβ42 uptake and promotes the degradation of internalized Aβ42 through clathrin-dependent or clathrin-independent endocytosis pathway.

5-7: Immunocytochemistry (ICC) and Microscopic Observations

The present inventors investigated whether ginsenosides can induce the expression of SRA, clathrin, caveolin 1, NEP or IDE and the differentiation of neurons actually through immunocytochemical analysis (ICC) and microscopic observations. Specifically, Cultured HMO6 cells were fixed in 4% paraformaldehyde (in 1×PBS) for 15 min, washed twice with 1×PBS supplemented with 100 mM glycine for 5 min, and incubated with permeabilization buffer consisting of 0.1% Triton X-100 (Sigma-Aldrich, USA) in 1×PBS for 30 min at room temperature. Blocking was performed with 1% bovine serum albumin (BSA, Sigma-Aldrich, USA) for 30 min at room temperature. Then, SRA, clathrin, caveolin1, NEP, or IDE mouse monoclonal antibody (1:200; Santa Cruz Biotechnology, USA) was added to 1% BSA in PBS with Tween 20 (PBST) and incubated for 2 h at room temperature. The cells were then washed three times with 1×PBS before fluorescein isothiocyanate (FITC)-conjugated or Alexa Fluor® 555-conjugated anti-mouse IgG 1:200 (Cell Signaling Technology, USA) was added to 1% BSA for 1 h at room temperature. The cells were rinsed and counterstained with 4′,6-diamidino-2-phenylindole (Sigma-Aldrich, USA) for 10 min, followed by two 1×PBS washes. Cultures were visualized with an inverted fluorescent microscope system (Eclipse Ti-S; Nikon, Japan) at a magnification of ×600.

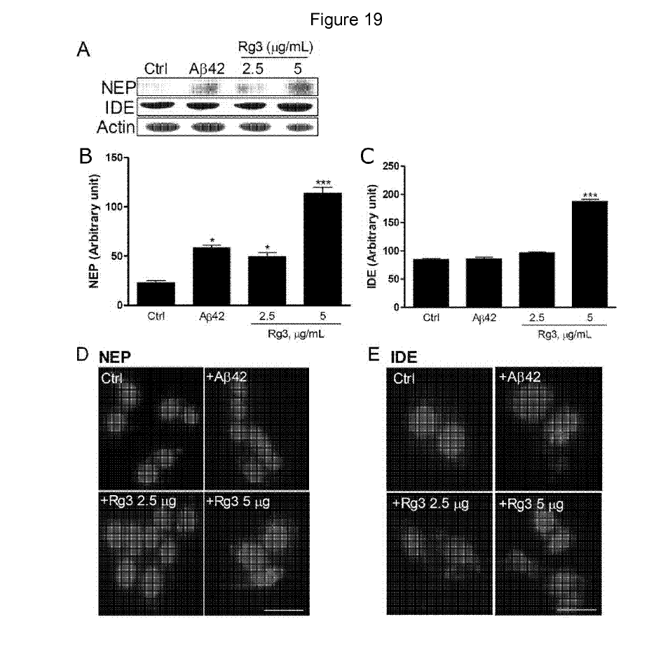

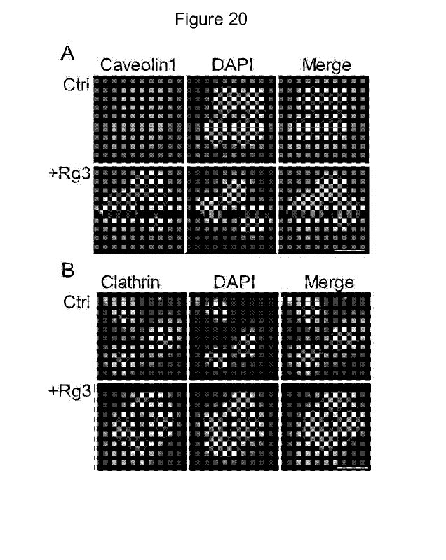

As shown in FIG. 19, western blots and ICC analysis coincided with the patterns of IDE and NEP gene expression (FIG. 19A-E). Interestingly, IDE and NEP were distinctively expressed in cells after treatment with 5 μg/ml Rg3 (FIGS. 19D and E). In addition, as shown in FIG. 20, Rg3-treated HMO6 cells highly expressed caveolin1 and clathrin. In conclusion, it was confirmed that the uptake of Aβ42 is induced by the caveolin- and clathrin-dependent endocytosis.

5-8: Uptake of FITC-Labeled Aβ in HMO6 Cells

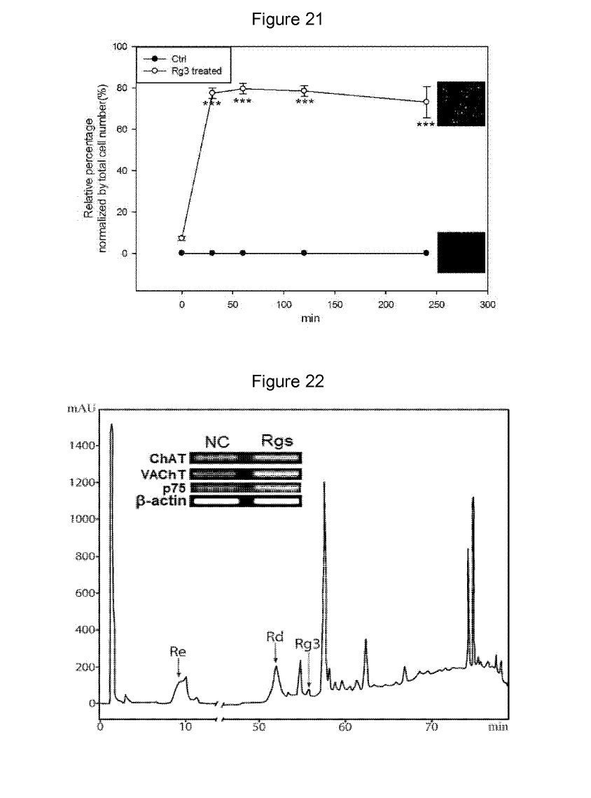

The stably transfected HM06.hSRA cells prepared in Example 5-5 were plated into 96-well plates at 5000 cells/well to analyze Aβ uptake. Cells were pre-treated with Rg3 (5 μg/ml) for 20 h and then the Aβ42 peptides were labeled with FITC using a FITC antibody labeling kit (Cat.#53027, Thermo Scientific, USA) and co-incubated for further 4 h. The kinetic measurement of Aβ42 uptake into HMO6 cytosol was evaluated using the IncuCyte™ FLR live-cell imaging system (Essen Bioscience, Hertfordshire, UK) every 30 min for 4 h under normal cell culture conditions. Fluorescence intensities represented the number of cells (count/mm²) over the time to quantify cells with FITC-labeled Aβ42 peptides. Aβ42 uptake was calculated using the following equation (FIG. 21):

$$A β 42 uptake ( \% ) = \frac{Total FITC positive cells}{Total number of live cells} \times 100$$

As shown in FIG. 21, Rg3-treated normal HMO6 cells increased Aβ42 uptake significantly under the live-cell imaging system after 30 min, which persisted for 6 h.

### Example 6

### Analysis of Passage of Rd, Re and Rg3 Through Blood-Brain Barrier

Alzheimer's disease is a representative brain disease, even a very potent therapeutic candidate should be administrated via more invasive method such as intracerobrospinal injection and intracranial injection if it cannot pass through blood-brain barrier (BBB), because it is impossible to deliver the therapeutic candidate to the brain via oral administration or a conventional parenteral administration such as intravenous injection and intramuscular injection.

Thus, it is a very important technical problem to screen active substance capable of passing through BBB or to formulate an active substance which does not pass through the BBB in a preparation capable of passing through the BBB in order to develop non-invasive general injectable therapeutics for treating Alzheimer's disease

6-1: HPLC Analysis

Thus, the present inventor performed an HPLC analysis on brain tissue extract obtained from experimental animals administrated with mixture of five ginsenosides (Rb1, Rd, Re, Rg1 and Rg3) which are contained in RgC, in order to investigate whether RgC or ginsenosides Rd, Re and Rg3 can pass through the BBB. For the HPLC analysis of ginsenosides transported into the brain, a mixture of Rb1, Re, Rd, Rg1, and Rg3 (10 mg/kg each) was administered to ten-week-old male BALB/c mice (Central Lab. Animals, Korea). Brain tissues were isolated six hours after the oral administration of the ginsenosides after the complete perfusion with 0.9% saline solution, mechanically disrupted using a TissueLyser (Qiagen, Crawley, U.K.), and centrifuged for 10 min at 13500 rpm. The supernatant (1 mL) was then mixed with n-butanol (0.4 mL) and the butanol layer was completely evaporated and dried for HPLC analysis. The chromatography was performed by the Agilent 1100 series HPLC system (Agilent Technologies, USA) equipped with a vacuum degasser, quaternary pump, and ultraviolet (UV) detector. Chromatographic separation was performed on a Zorbax SB-Aq C18 column (4.6150 mm, 5 μm) and a mixture of solvent A (water) and solvent B (acetonitrile) flowed at a rate of 1 mL/min. The gradient elution program was initiated with 79% solvent A and 21% solvent B, 0-10 min (21-22% B), 10-11 min (22-23% B), 11-29 min (23-24% B), 29-34 min (24-30% B), 34-44 min (30-32% B), 44-49 min (32-50% B), 49-64 min (50-65% B), 64-78 min (65-100% B), and equilibrated with the initial gradient program (21% B) for 10 min prior to the next injection. The column temperature was set at 35° C., the volume of the sample injection was 10 μL, and detection wavelength was set at 203 nm. The animal experiments were approved by the Gangneung-Wonju National University Animal Care and Use Committee (Approval Number: GWNU-2013-23), and all procedures were conducted in accordance with the Guide for Care and Use of Laboratory Animals published by the National Institute of Health. As a result shown in FIG. 22, it was confirmed that three ginsenosides (Rd, Re and Rg3) among five species penetrated into the brain.

6-2: Analysis of In Vivo Expression of AChT and VAChT

In addition, the present inventor analyzed the expression level of ChAT and VAChT mRNA of brain tissues taken from mice 6 h after administrating five ginsenosides orally using quantitative RT-PCR similar with the method of Example 4-2-1, in order to determine whether the RgC in accordance with an embodiment of the present invention increases the expression level of AChT and VAChT in the brain of experimental animals actually.

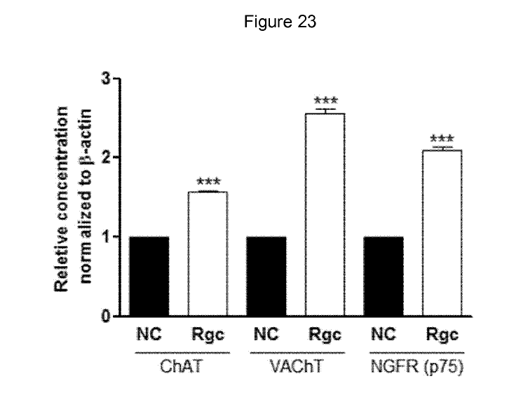

As a result shown in FIG. 23, it was confirmed that there was a remarkable increase in ChAT and VAChT mRNA from the same brain tissue six hours after oral administration of five ginsenosides. Interestingly, another cholinergic gene, p75, was significantly increased in the brain cortex as well.

The result proves that the RgC in accordance with an embodiment of the present invention is a very effective substance capable of improving decreased cognitive function which is a major symptom of Alzheimer's disease by increasing the expression level of ChAT and VAChT and raising the level of acetylcholine, a neurotransmitter in the brain thereby even via a non-invasive oral administration.

6-3: Analysis of In Vivo Expression of SRA and SRB

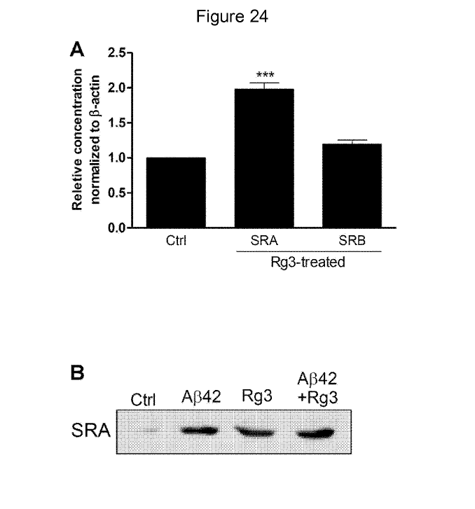

The present inventor investigated whether ginsenoside Rg3 translocated to brain tissues contributes to the uptake and degradation of Aβ42 in order to determine whether in vitro experimental results described in the Example 5 can be applied to in vivo condition in the same manner. Specifically, the expression level of SRA and SRB gene within brain tissues of mice administrated with Rg3 was determined using the RT-PCR analysis described in the Example 5-4 (FIG. 24A) and the western blot analysis described in the Example 5-6 (FIG. 24B). As a result shown in FIGS. 24A and 24B, 24B, it was confirmed that the expression of SRA mRNA and protein in brain tissues was increased when Rg3 was administrated to animals. This result suggests that ginsenoside Rg3 and RgC containing the same as a major component in accordance with an embodiment of the present invention can be used for the treatment of diseases caused by the accumulation of beta-amyloid such as Alzheimer's disease via oral administration.

### Example 7

### Experiments Related to Improvement of Cognitive Function

The present inventor performed learning and memory tests using rats in order to investigate whether Rg3 can ameliorate symptoms of Alzheimer's model animals in deed. Particularly, passive avoidance trials (PAT) were performed once per day for 4 consecutive days with a fifth trial 1 week after the fourth trial to evaluate memory acquisition and retention. The latency time of stay in the light room from lights-on was recorded following an electric shock (1 mA for 2 s) in the dark compartment. Morris water maze tests were performed in a circular water bath filled with water maintained at 22±2° C. The bath was divided into four quadrants, and a hidden escape platform (10 cm in diameter) was submerged in the center of one quadrant. The rats were trained to find the hidden platform, based on several cues external to the maze. Three trials were conducted on each day with 5-min intervals for the 4 consecutive days followed by a fifth performance 1 week after the day 4 trial. The mean time spent to escape onto the platform was recorded.

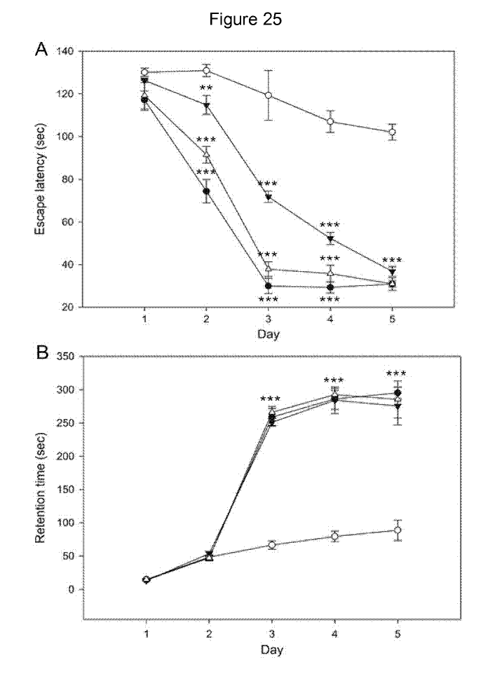

Aa s result shown in FIGS. 25A and 25B, it was confirmed that rats infused with Aβ42 displayed severely impaired learning and memory functions as measured by both the Morris water-maze (FIG. 25A) and passive avoidance (FIG. 25B) performance during the trials. However, Aβ42 rats administered Rg3 maintained learning and memory functions at 4-5 weeks, indicating that Rg3 prevents the brain from injurious Aβ42 prior to attacking neuronal cells.

The data are represented as mean±SD and statistical comparisons between the groups were performed using one-way ANOVA with a Dunnet's post-hoc test using SPSS v. 13 software (SPSS, USA) and statistical significance was set a priori at p<0.05.

In conclusion, the present invention demonstrates that Rg3, a major component of RgC may prevent AD pathogenesis by enhancing SRA-mediated Aβ42 uptake, clathrin- and caveolae-dependent Aβ42 endocytosis, and NEP/IDE-mediated Aβ42 degradation. Thus, Rg3 and RgC containing the same could be a promising anti-AD therapeutic candidate in terms of AD-specific biochemical and physiological functions of Rg3.

As described above, the ginsenoside complex (RgC) in accordance with an embodiment of the present invention improved cognitive function of Alzheimer's model animals by eliminating aggregative amyloid beta (Aβ42) related to pathology of Alzheimer's disease in the model animals due to the function of ginsenoside Rg3 as well as increasing the expression of ChAT and VAChT related to cognitive function in Alzheimer's model animals due to the action of ginsenosides Rd and Re. Therefore, it can be acknowledged that the RgC according to an embodiment of the present invention is a highly effective therapeutic candidate having a synergistic effect with dual activities of inhibiting the neuronal cell death caused by amyloid plaques and improving cognitive function.

Further, since the RgC in accordance with an embodiment of the present invention is manufactured from ginseng berries which have been abandoned rather than a ginseng root, a major part to be used thus it may contribute to income increase of ginseng raising farms, and industrial value thereof would be very large.

Although the invention has been described by reference to the above described examples, which is only as illustrative, those skilled in the art will appreciate that various modifications and equivalent other embodiments are possible. Therefore, the true scope of the present invention should be defined by the technical features of the appended claims.

1. A method for preparing a ginsenoside complex effective for Alzheimer's disease, the method comprises:
i) preparing steam-dried unripened ginseng berries by steaming and drying unripened ginseng berries 3 to 10 times;
ii) preparing an extract of steam-dried ginseng berries by extracting the streamed-dried ginseng berries with water, C1 to C4 alcohol or a mixture thereof;
iii) fractionating the extract of steam-dried ginseng berries to an ethyl acetate layer and a water layer by adding a solvent comprising ethyl acetate and water to the extract; and
iv) preparing a n-butanol fraction by further fractionating the water layer with n-butanol to form a n-butanol layer and recrystallizing the n-butanol layer with ether.

2. The method according to claim 1, wherein the alcohol is methanol, ethanol or propanol.

3. The method according to claim 1, wherein the ginsenoside complex comprises at least 4 wt % of ginsenoside Re, at least 8 wt % of ginsenoside Rd and at least 20 wt % of ginsenoside Rg3.

4. The method according to claim 1, further comprising isolating the fractions corresponding to Rf value of 0.4 to 0.7 on a thin layer chromatography (TLC) using running a solvent of ethyl acetate:methanol=3:1 from eluted fractions of the n-butanol fraction by the chromatography.

5. A pharmaceutical composition for treating Alzheimer's disease or improving cognitive function in a patient suffering from Alzheimer's disease comprising the ginsenoside complex prepared by the method of claim 1 and a pharmaceutically acceptable carrier.

6. A pharmaceutical composition for treating Alzheimer's disease or improving cognitive function in a patient suffering from Alzheimer's disease comprising the ginsenoside complex prepared by the method of claim 4 and a pharmaceutically acceptable carrier.

7. A method for treating or improving cognitive function in a subject suffering from Alzheimer's disease comprising administrating a therapeutically effective amount of the ginsenoside complex prepared by the method of claim 1 to the subject.

8. The method according to claim 7, wherein the ginsenoside complex comprises at least 4 wt % of ginsenoside Re, at least 8 wt % of ginsenoside Rd and at least 20 wt % of ginsenoside Rg3.

9. A method for treating or enhancing cognitive activity in a subject suffering from Alzheimer's disease comprising administrating a therapeutically effective amount of the ginsenoside complex prepared by the method of claim 4 to the subject.


SEQUENCE LISTING (42 sequences)

SEQ ID NO: 1 - DNA, 20 bp, Artificial Sequence
  ChAT forward primer
gttccccagaaactcaaggc

SEQ ID NO: 2 - DNA, 20 bp, Artificial Sequence
  ChAT reverse primer
gagtggccgatctgatgttg

SEQ ID NO: 3 - DNA, 20 bp, Artificial Sequence
  VAChT forward primer
ttgatcgcatgagctacgac

SEQ ID NO: 4 - DNA, 20 bp, Artificial Sequence
  VAChT reverse primer
ccactaggcttccaaagctg

SEQ ID NO: 5 - DNA, 20 bp, Artificial Sequence
  APP forward primer
gtggtccgagttcccacgac

SEQ ID NO: 6 - DNA, 20 bp, Artificial Sequence
  APP reverse primer
tcataacctgggaccggatc

SEQ ID NO: 7 - DNA, 20 bp, Artificial Sequence
  MAP2 forward primer
accacacctgcagtggagaa

SEQ ID NO: 8 - DNA, 20 bp, Artificial Sequence
  MAP2 reverse primer
aatctggacctggttcctgc

SEQ ID NO: 9 - DNA, 20 bp, Artificial Sequence
  p21 forward primer
agtgtgccgttgtctcttcg

SEQ ID NO: 10 - DNA, 20 bp, Artificial Sequence
  p21 reverse primer
tcaaagttccaccgttctcg

SEQ ID NO: 11 - DNA, 20 bp, Artificial Sequence
  TrkA forward primer
ccagatgcccaatgactctg

SEQ ID NO: 12 - DNA, 20 bp, Artificial Sequence
  TrkA reverse primer
cccagcacgtcacattcttc

SEQ ID NO: 13 - DNA, 20 bp, Artificial Sequence
  NGF forward primer
tactgcaccaatagctgccc

SEQ ID NO: 14 - DNA, 20 bp, Artificial Sequence
  NGF reverse primer
tccggtgagtcctgttgaaa

SEQ ID NO: 15 - DNA, 20 bp, Artificial Sequence
  NGFR forward primer
aagagatccctggtcgatgg

SEQ ID NO: 16 - DNA, 20 bp, Artificial Sequence
  NGFR reverse primer
gcagccaagatggagcaata

SEQ ID NO: 17 - DNA, 20 bp, Artificial Sequence
  CHT1 forward primer
tctgcagtacctctgccctg

SEQ ID NO: 18 - DNA, 20 bp, Artificial Sequence
  CHT1 reverse primer
gctccgaacacaagcacagt

SEQ ID NO: 19 - DNA, 19 bp, Artificial Sequence
  hSRA forward primer
ggagatcgaggtcccactg

SEQ ID NO: 20 - DNA, 20 bp, Artificial Sequence
  hSRA reverse primer
tgtttccactccccttttcc

SEQ ID NO: 21 - DNA, 20 bp, Artificial Sequence
  hSRB forward primer
accctaaccaggaggcacac

SEQ ID NO: 22 - DNA, 20 bp, Artificial Sequence
  hSRB reverse primer
gtgaagagtctccccctcca

SEQ ID NO: 23 - DNA, 20 bp, Artificial Sequence
  mSRA forward primer
ttcactggatgcaatctcca

SEQ ID NO: 24 - DNA, 20 bp, Artificial Sequence
  mSRA reverse primer
acgtgcgcttgttcttcttt

SEQ ID NO: 25 - DNA, 20 bp, Artificial Sequence
  mSRB forward primer
acaaatggaacggactcagc

SEQ ID NO: 26 - DNA, 20 bp, Artificial Sequence
  mSRB reverse primer
gtgaagagtctccccctcca

SEQ ID NO: 27 - DNA, 20 bp, Artificial Sequence
  NEP forward primer
atcagcctctcggtccttgt

SEQ ID NO: 28 - DNA, 20 bp, Artificial Sequence
  NEP reverse primer
tggaagacagcgcaagactc

SEQ ID NO: 29 - DNA, 20 bp, Artificial Sequence
  IDE forward primer
cagtgatcccaccacggata

SEQ ID NO: 30 - DNA, 20 bp, Artificial Sequence
  IDE reverse primer
gtgccctagacaggtttgca

SEQ ID NO: 31 - DNA, 20 bp, Artificial Sequence
  MMP9 forward primer
ctcctactctgcctgcacca

SEQ ID NO: 32 - DNA, 20 bp, Artificial Sequence
  MMP9 reverse primer
aactacgaccgggacaagct

SEQ ID NO: 33 - DNA, 20 bp, Artificial Sequence
  caveolin1 forward primer
cagacgagctgagcgagaag

SEQ ID NO: 34 - DNA, 20 bp, Artificial Sequence
  caveolin1 reverse primer
gacagcaagcggtaaaacca

SEQ ID NO: 35 - DNA, 20 bp, Artificial Sequence
  caveolin2 forward primer
agccatgccctctttgaaat

SEQ ID NO: 36 - DNA, 20 bp, Artificial Sequence
  caveolin2 reverse primer
acgctcgtacacaatggagc

SEQ ID NO: 37 - DNA, 20 bp, Artificial Sequence
  clathrin forward primer
aagcccttgatgccaattct

SEQ ID NO: 38 - DNA, 20 bp, Artificial Sequence
  clathrin reverse primer
agatcgttcgtccggtttct

SEQ ID NO: 39 - DNA, 20 bp, Artificial Sequence
  GAPDH forward primer
ggagccaaaagggtcatcat

SEQ ID NO: 40 - DNA, 20 bp, Artificial Sequence
  GAPDH reverse primer
accacagtccatgccatcac

SEQ ID NO: 41 - DNA, 20 bp, Artificial Sequence
  beta-actin forward primer
tacagcttcaccaccacagc

SEQ ID NO: 42 - DNA, 20 bp, Artificial Sequence
  beta-actin reverse primer
aaggaaggctggaaaagagc